(19) United States
(12) Patent Application Publication
(10) Pub. No.: US 2011/0008409 A1
(43) Pub. Date: Jan. 13, 2011

(54) PHARMACEUTICAL PREPARATION CONTAINING A GESTAGEN, AND KIT AND METHOD FOR TREATING ENDOMETRIOSIS USING THE PREPARATION

(75) Inventors: Christian Seitz, Zeuthen (DE); Annemarie Wasserfall, Berlin (DE); Holger Zimmermann, Falkensee (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Bayer Schering Pharma AG, Berlin (DE)

(21) Appl. No.: 12/885,919

(22) Filed: Sep. 20, 2010

Related U.S. Application Data
(62) Division of application No. 12/037,990, filed on Feb. 27, 2008.
(60) Provisional application No. 60/892,393, filed on Mar. 1, 2007.

(51) Int. Cl. A61F 6/08; A61K 31/57; A61K 9/36; A61P 35/00

(52) U.S. Cl. 424/430; 514/179; 424/479

(57) ABSTRACT

The pharmaceutical preparation for treating endometriosis contains at least 28, preferably 30, daily dose units, each of which contain dienogest, cyproterone acetate, or chlormadinone acetate at a daily dose that is at most twice that required to inhibit ovulation together with one or more pharmaceutical aids and/or carriers. The daily dose units are administered in a method of prophylaxis and/or therapy of endometriosis continuously during a time interval of at least 169 days or 25 weeks, preferably more than two years. The method effectively reduces endometriosis and associated pain, while undesirable side effects including bone density decrease are reduced or eliminated.

CROSS-REFERENCE This is a continuation of U.S. Provisional Patent Application Ser. No. 60/892,393, of Mar. 1, 2007. The aforesaid U.S. Provisional Patent Application describes the same invention that is disclosed and claimed herein below and provides the basis for a claim of priority of invention under 35 U.S.C. 119 (e).

### BACKGROUND OF THE INVENTION

1. The Field of the Invention The invention relates to a pharmaceutical preparation for reducing endometriosis that contains a gestagen with anti-androgenic activity in a daily dose amounting to at the most or up to twice the ovulation-inhibiting dose, together with one or more pharmaceutically acceptable aids and/or carriers. The invention also relates to a monophasic preparation that exerts no negative effect on the bone metabolism. Hence, this preparation is suitable for long-term use. The use of gestagens with androgenic action at the above-indicated dose, however, makes it possible to produce a pharmaceutical preparation for prophylaxis and/or therapy of endometriosis while keeping the known side effects, for example hot flashes, acne, and changes in the lipid profile, to an acceptable level.

2. Description of the Related Art

Endometriosis is a chronic, gynecological disease affecting primarily 5-20% of women of childbearing age. In the technical literature, endometriosis is defined as the appearance of the endometrium or endometrium-like tissue outside the uterine cavity. Typical symptoms of endometrial disease are dysmenorrhea, dyspareunia, and painful bowel movement. Endometriosis patients often complain of pain in the pelvic region. Endometrial disease is often suggested by lower abdomen pain appearing in the second half of the anovulatory cycle, followed by painful menstrual bleeding, followed by freedom from discomfort until the middle of the next cycle. Alternatively persistent pain is not rare. At any rate, about 30-40% of endometriosis patients have no discomfort. The disease is then detected accidentally in connection with other diagnostic measures. In about 50-60% of the subjects, a diagnosis of “endometriosis disease” is made as an accidental diagnosis to explain sterility.

It is known from the technical and patent literature to treat endometriosis with drugs such as Danazol, a derivative of 17α-ethinyltestosterone, GnRH [gonadotropin-releasing hormone] agonists, gestagen/estrogen combinations or preparations based on only gestagen.

U.S. Pat. No. 6,569,845 discloses the treatment of angiogenic diseases with dienogest at a daily dose of 0.5 to 10 mg. Corresponding pharmaceutical preparations indicated as examples, and which could be used extensively also for the treatment of endometriosis, have dienogest content of from 400 mg to 2 g.

Moore, C., et al, in The Treatment of Endometriosis, Drugs of Today 1999, 35 (Suppl. C): pp. 41-52, studied in clinical investigations the efficacy of dienogest in the treatment of endometriosis comparatively with the treatment regime of Danazol or GnRH agonists. The subjects affected with endometriosis were given 2 mg of dienogest per day for 24 weeks. The result of the treatment was comparable with that of a treatment with Danazol or GnRH agonists. Up to 90% of the affected subjects reported irregular bleeding, but none reported unbearable bleeding. The efficacy of the standard treatment with Danazol was reduced by significant androgenic effects, whereas the GnRH agonists brought about “menopausal symptoms”.

Schweppe, K. W., “Stellenwert der Gestagene” [Importance of the Gestagens], Zentralbl. Gynaekol. 2003, 125: pp. 276-280, states that low estrogen levels were recorded during continuous oral gestagen treatment (for example with medroxy-progesterone acetate, dienogest, dihydrogesterone, or lynesterenol at a daily dose from 5 to 20 mg, characterized as a low dose and classified as effective treatment principle in case of endometriosis-induced symptoms). Spotting and intracyclic menstrual bleeding often resulted. This requires an increase in dosage and/or estrogen addition. Over a long period of time, long-term relapse rates amount to more than 50%.

Safety information from 2005 relating to a medroxyprogesterone acetate product indicates that preparations based on gestagen alone can exert a negative effect on bone density, particularly as a result of long-term treatment. In combination with estrogens, on the other hand, gestagens have a positive effect on bone metabolism.

Another safety information pamphlet, NDA 21-584, FDA of Mar. 22, 2005, for DEPOSUBQ PROVERA 104™ (medroxyprogesterone acetate i.m.—104 mg/0.65 mL) points out that women using this preparation become affected with bone mineral density loss which progresses with the duration of use of the preparation and is no longer completely reversible.

Knauthe, R., and Habenicht, U. F., in “Levonorgestrel has Beneficial Effects”, Exp. Clin. Endocrinol. Diabetes 106 (1998), Suppl. 1:37, pointed out as early as 1998 that in this case the partial androgenic activity of a gestagen (levonorgestrel) and not the gestagenic activity is responsible for this positive effect on the bone metabolism. Kuhl, H., “Klimakterium, Postmenopause and Hormonsubstitution” [Menopause, Postmenopause and Hormone Replacement], 3rd. ed., Bremen,

UNI-MED, 2006, 117, also stresses that certain gestagens become active by way of their androgenic partial activity. Moreover, Kuhl states that androgens markedly enhance the positive effect of estrogens on bone density.

### SUMMARY OF THE INVENTION

The object of the invention is a pharmaceutical composition with as low a steroidal content as possible for reducing endometriosis, the composition at the same time having no negative effect on the bone density/bone metabolism.

We have now found that endometriosis can be reduced with the aid of a pharmaceutical preparation of low hormonal dosage, which comprises a daily dose of a gestagen with anti-androgenic activity, which is at the most or no more then twice the dose required to inhibit ovulation, and one or more pharmaceutically acceptable aids and/or carriers.

At the same time, the pharmaceutical preparation, or the method of treatment using the preparation, or the corresponding monophasic preparation, besides reducing endometriosis, exerts no negative effect on the bone metabolism so that no reduction or decrease in bone density is observed.

At the same time, surprisingly, the pharmaceutical preparation, or the method of treatment using the preparation, or the corresponding monophasic preparation, keeps the known side effects, for example hot flashes and changes in the lipid profile, which are caused by the conventional drugs for treating endometriosis within tolerable limits.

According to the invention, the gestagen with anti-androgenic activity contained in the pharmaceutical preparation for treating endometriosis, or used as the effective ingredient in the method of treatment for endometriosis, is 17α-cyanomethyl-17-β-hydroxyestra-4, 9-dien-3-one (dienogest), cyproterone acetate, or chiormadinone acetate.

The daily dose of dienogest is at the most, or up to, twice the ovulation-inhibiting dose and amounts at most to 2 mg. According to the invention, cyproterone acetate or chlormadinone acetate are used at a daily dose of at most twice the ovulation-inhibiting dose. For the purposes of the present invention the ovulation-inhibiting dose of cyproterone acetate is 1 mg and that of chiormadinone acetate is 1.7 mg.

The daily dose of the gestagens can be equal at most to two times the ovulation-inhibiting dose, or at most to two times one-half of the ovulation-inhibiting dose.

According to the invention, the objective of the invention is also attained by a pharmaceutical preparation containing separately packaged and individually removable daily dose units sufficient for a period of 28 or 30 consecutive days and placed in a package unit and also be a method of using this pharmaceutical preparation. The daily dose units each contain a maximum of 2 mg of dienogest, an equivalent amount of cypropterone acetate, or an equivalent amount of chiormadinone acetate, together with one or more pharmaceutically acceptable aids and/or carriers. The package units preferably consist of two blisters with 14 or 15 daily dose units in each blister.

Surprisingly, we have found that the pharmaceutical preparation according to the invention, which is suitable for prophylaxis and/or therapy of endometriosis, has no negative effect on bone metabolism and bone density nor on the lipid profile. Hence, surprisingly, the pharmaceutical preparation is suitable for long-term administration, particularly continuous administration, of the dose units for a period from at least 169 days or at least 25 weeks, preferably several years, for example more than 2 years.

According to the invention, the objective is also attained by a kit containing at least 28, preferably 30, daily dose units of at the most twice the ovulation-inhibiting dose of a gestagen with partial androgenic activity, preferably dienogest, cypropterone acetate, or chlormadinone acetate, together with one or more aids and/or carriers that are pharmaceutically acceptable.

Moreover, the present invention relates to a method of treating, particularly for propholaxis and/or therapy, of endometriosis, which comprises using gestagens with anti-androgenic activity at a daily dose amounting to at the most or no more than twice the ovulation-inhibiting dose, and also to a method of producing a pharmaceutical preparation for the prophylaxis and/or therapy of endometriosis. It has been surprisingly found that the aforesaid pharmaceutical preparation according to the invention exerts no negative effect on bone metabolism so that no reduction in bone density is observed. At the same time, the known side effects of the conventional drugs for treating endometriosis, for example hot flashes and changes in the lipid profile, are kept within bearable limits.

The gestagen used in the method of treating endometriosis according to the invention is 17α-cyanomethyl-17-β-hydroxyestra-4, 9-dien-3-one (dienogest), cyproterone acetate, or chlormadinone acetate. The gestagen is preferably administered daily at a daily dose that is effective for reducing or preventing endometriosis while keeping side effects within bearable limits, particularly 2 mg in the case of dienogest. Alternatively an equivalent amount of cyproterone acetate or chlormadinone acetate is administered daily. The daily dose of the gestagen is preferably administered continuously for at least 169 days or 25 weeks to several years, preferably more than two years, in the method of treating endometriosis.

Surprisingly the method of treating endometriosis according to the invention has no negative effect on bone metabolism and thus does not reduce bone density.

The pharmaceutical preparation according to the invention for treating endometriosis can be in the form of tablets, capsules, sugar-coated tablets, wafers, transdermal therapy systems, ampoules, suppositories, gels, ointments, implants, vaginal rings, or nasal sprays. The daily gestagen dose released by the non-oral forms of the pharmaceutical preparation, such as transdermal therapy systems, ampoules, suppositories, gels, ointments, implants, vaginal rings, or nasal sprays, should be equivalent to the daily dose unit amounting to at the most, or up to, twice the ovulation-inhibiting dose present in the oral forms.

Furthermore, the invention relates to a monophasic preparation for reducing Endometriosis, which comprises at least 28 dose units, preferably 30 dose units, optionally in two blisters each of which contain 14 or 15 dose units. Each dose unit contains a dose of a gestagen with anti-androgenic activity, which is at most twice the dose required to inhibit ovulation and which is selected from the group consisting of dienogest, cyproterone acetate and chiormadinone acetate.

The invention also relates to a monophasic preparation for reducing endometriosis, which comprises the aforesaid dose units, each containing a dose of a gestagen with anti-androgenic activity, which is at the most twice the ovulation-inhibiting dose and which is selected from the group consisting of dienogest, cyproterone acetate and chlormadinone acetate. These dose units are continuously administered daily for from 169 days to more than 730 days.

The monophasic preparation is suitable for prophylaxis and/or therapy of endometriosis or reduces endometriosis, but does not exert a negative effect on the bone metabolism/bone density, while keeping the known endometriosis therapy-induced side effects (reduced bone density, hot flashes, changed lipid profile) within bearable limits. For this reason, it is suitable for long-term therapy.

### PRACTICAL EXAMPLES

### Example 1

Tablets having the following composition were prepared:

| | |
|---|---|
| Dienogest, micronized | 2.000 mg |
| min. 99% ≦ 20 μm, 100% < 30 μm | |
| Lactose monohydrate | 62.800 mg |
| Microcrystalline cellulose | 18.000 mg |
| Potato starch | 36.000 mg |
| Povidone K 25 | 8.100 mg |
| Magnesium stearate | 1.350 mg |
| Talc | 4.050 mg |
| Crospovidone | 2.700 mg |

Dienogest was micronized to an average particle size of 20 μm and used in admixture with lactose monohydrate, microcrystalline cellulose and potato starch. Povidone K 25 was sprayed in during the granulation. After drying and addition of talc, crospovidone and magnesium stearate, the mixture of the substances was compressed into tablets with a diameter of 7 mm and weighing 135 mg.

### Example 2

In a clinical study, 252 women with laparoscopically diagnosed endometriosis were treated over a period of 6 months either with the GnRH agonist leuprorelin acetate (LA), 3.75 mg s.c. every 4 weeks, or orally with 2 mg/d of the gestagen dienogest (DNG). 128 patients were randomly assigned to the LH group and 124 to the DNG group. The efficacy of each therapy was evaluated by means of, among other methods, a pain scale (visual analog scale, VAS) form filled out by the patient. At the end of the treatment, similar pain reduction was noted in the two comparative groups compared to the pain experienced at the beginning of therapy (−47.5 mm for DNG; −46.0 mm for LA). Statistical analysis showed that DNG was not inferior to LA.

Moreover, the subjects with endometriosis showed frequent side effects of hormonal therapy methods.

In both treatment groups, changes in menstrual bleeding took place—often in the form of an absence of regular bleeding or in the form of slight intracyclic menstrual bleeding—but caused only few patients to discontinue therapy.

Hot flashes, a typical symptom of estrogen deficiency, occurred in the DNG group substantially more rarely (0.89 days with hot flashes/week) than in the LA group (4.23 days/week). In addition, the symptomatology in the DNG group was reduced in the course of the 6-month therapy, whereas in the LA group it increased.

The effect of the two therapies on bone metabolism was studied in a subgroup of patients. At the end of the 6-month therapy, a statistically significant difference was seen to the advantage of DNG: Under DNG, the bone density was nearly unchanged compared to that noted at the beginning of the study (+0.25%) whereas in the comparative group a marked decrease took place (−4.0%). These results were confirmed by laboratory parameters for determining bone metabolism, which indicated increased bone resorption under therapy with LA.

Under therapy with DNG, the estrogen values remained essentially unchanged (average value before therapy: 256.3 pmole/L; at the end of study: 249.9 pmole/L) whereas under LA an appreciable decrease took place (before therapy: 299.0 pmole/L; at the end of study: 68.5 pmole/L). The other laboratory values concerning safety showed no significant changes in the two treatment groups.

Overall, the two treatments were equivalent as regards efficacy whereas in terms of side effects caused by estrogen deficiency such as hot flashes and reduced bone density, DNG showed marked advantages.

While the invention has been illustrated and described as embodied in a pharmaceutical preparation containing a gestagen, a kit, and a method for treating endometriosis using this pharmaceutical preparation, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1-24. (canceled)

25. A method of treating endometriosis, said method comprising administering a pharmaceutical composition comprising dienogest by daily administration for at least 25 weeks in a daily dose amounting to up to twice the dose effective for inhibiting ovulation.

26. The method of claim 25, wherein said daily dose is 2 mg of said dienogest.

27. The method of claim 25, wherein the composition is administered by daily administration for more than 2 years.

28. The method of claim 25, comprising administering said daily dose to an individual suffering from endometriosis in order to reduce said endometriosis.

29. The method of claim 25, comprising administering said daily dose to an individual to prevent occurrence of endometriosis.

30. The method of claim 25, wherein said method results in no negative effect on bone metabolism and does not reduce bone density.

31. The method of claim 25, wherein said pharmaceutical composition further comprises one or more pharmaceutically acceptable aids and/or carriers and is in the form of a tablet, capsule, sugar-coated tablet, wafer, transdermal therapy system, ampoule, suppository, gel, ointment, implant, vaginal ring or nasal spray.

32. The method of claim 25, wherein said pharmaceutical composition is provided in the form of package units containing at least 28 daily dose units, each dose unit being separately packaged and individually removable.

33. The method of claim 32, wherein the daily dose units each comprise 2 mg of dienogest and one or more pharmaceutically acceptable aids and/or carriers.

34. The method of claim 33, wherein each package unit contains 30 of said separately packaged and individually removable daily dose units.
